(19) United States
(12) Patent Application Publication
(10) Pub. No.: US 2011/0237658 A1
(43) Pub. Date: Sep. 29, 2011

(54) COMPOUND AND METHOD FOR THE TREATMENT OF PAIN

(75) Inventor: MAHESH KANDULA, Medapadu (IN)

(73) Assignee: KRISHANI BIOSCIENCES (P) LTD, Hyderabad (IN)

(21) Appl. No.: 13/046,721

(22) Filed: Mar. 12, 2011

Related U.S. Application Data
(60) Provisional application No. 61/316,515, filed on Mar. 23, 2010.

(51) Int. Cl. A61K 31/385; C07D 339/04; A61P 29/00

(52) U.S. Cl. 514/440; 549/39

(57) ABSTRACT

The disclosure herein provides a compound of formula 1. The disclosure also provides a method of synthesizing the compound of formula 1. The compound of formula 1 or its pharmaceutical acceptable salts, as well as polymorphs, solvates, and hydrates thereof may be formulated as pharmaceutical composition. The pharmaceutical composition of compound of formula 1 or the final compound may be formulated for non-invasive peroral, topical (example transdermal), enteral, transmucosal, targeted delivery, sustained release delivery, delayed release, pulsed release and parenteral methods. Such compositions may be used to treat chronic pain manifested with chronic diseases or its associated complications.

### CROSS REFERENCE TO RELATED APPLICATION

This application claims priority to U.S. Provisional Patent Application No. 61/316,515 filed on Mar. 23^{rd}, 2010. This application is hereby incorporated by reference in its entireties for all of its teachings.

### TECHNICAL FIELD

This disclosure generally relates to compound, method of synthesizing the compound and method for the treatment of pain. More particularly, this disclosure relates to treating subjects suffering from neuropathic pain with pharmaceutically acceptable dose of compound or the prodrug.

### BACKGROUND

Pain attributed to tissue injury is mainly caused by inflammation. The mechanism of peripheral inflammation includes local liberation of mediators released by cell lysis, inflammatory cells, and nerve endings. Nerve roots are vulnerable to compression (e.g., compressive radiculopathy, infections, and tumors). If the lesion is proximal to the dorsal root ganglion, there may be abnormality of the central axons but not necessarily of the peripheral axons. Therefore, tests aimed at the peripheral axons will not detect the injury in those situations. Likewise, complete degeneration of the axon is not necessary to produce clinical symptoms: lesions may be in the form of perinodal retraction of myelin or frank demyelination. Demyelination with ephaptic spread of action potentials between adjacent axons is believed to underlie bursts of lacerating pain because the action potentials transmitted along a few fibers can inappropriately spread many other axons.

Chronic pain is a significant global health, economic and social problem. Complex regional pain syndrome is one of the most severe and mysterious neuropathic pain syndromes. The clinical symptoms of complex regional pain syndrome always include pain, hyperalgesia, and allodynia.

Managing acute and chronic pathology of pain often relies on the addressing underlying pathology and symptoms of the disease. There is currently a need in the art for new compounds for treatment of acute and chronic pain.

### SUMMARY OF DISCLOSURE

The instant disclosure presents a compound of formula 1, method of synthesizing the compound of formula 1 and using the compound of formula 1 for treating a mammal suffering with pain. In one embodiment a pharmaceutical composition comprising one or more compounds of formula 1 or intermediates' thereof with one or more of pharmaceutically acceptable carriers, vehicles or diluents are disclosed and used for treating pain. In another embodiment, these compounds may be used in the treatment of pain and related complications.

In one embodiment, a compound of formula 1 is disclosed.

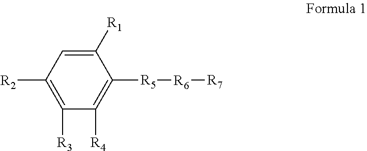

Wherein, R1, R2, R3 and R4 each independently may represent hydrogen, methyl, amine, cyclohexyl methyl ether, butoxy, propoxy, halogen (Chlorine or Flourine), thiol, alkyl, alkyl thiol, acetyl thiol, disulfide, acyl, acylalkyl, alkenyl, alkylthioalkyl, alkynyl, alkoxyaryl, alkoxyalkyl, aryl, aralkyl, aryloxyalkyl, arylthioalkyl, cycloalkyl, ether, ester, heteroaryl, heterocyclyl, lower alkyl, sulfone, sulfoxide, or hydroxyalkyl;
R5 independently represents hydrogen, carboxyl, amine, —NH—CO—NH—, —NH—CO—CH2—NH—,

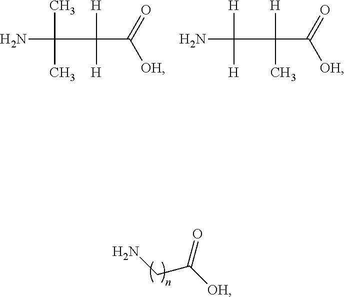

or an analog of any of the foregoing; where, n represents an integer from 0 to 11.
R6 independently represents hydrogen, amine, —NH—CO—, R—COO—R1, thiol, disulfide, or

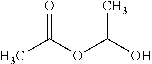

R7 independently represents at least one of a 6,8-dithiooctanoic acid,

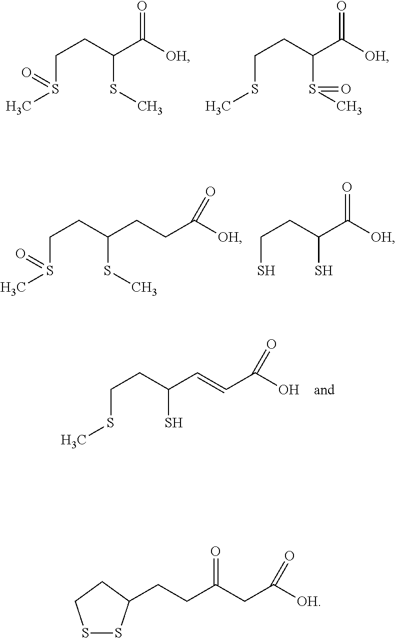

In one embodiment, R1, R2, R3 or R4 may represent a hydrogen, methyl, amine, butoxy, propoxy, chlorine or

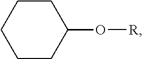

R5 represents —NH—CO—NH—, —NH—CO—CH2—NH— or —COO—(CH2)—NH—, R6 represents

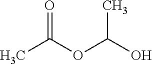

and R7 represents R-isomer of residue or analog or derivative or metabolite of 6,8-dithiooctanoic acid.

In certain embodiments, the compounds and compounds of formula (I) or pharmaceutically acceptable salts thereof,

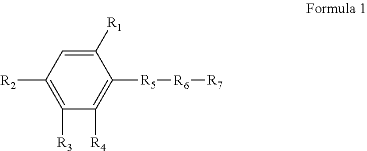

Wherein, R1, R2, R3 and R4 each independently represents hydrogen, methyl, amine, cyclohexyl methyl ether, butoxy, propoxy, halogen (Chlorine or Flourine), thiol, alkyl, alkyl thiol, acetyl thiol, disulfide, acyl, acylalkyl, alkenyl, alkylthioalkyl, alkynyl, alkoxyaryl, alkoxyalkyl, aryl, aralkyl, aryloxyalkyl, arylthioalkyl, cycloalkyl, ether, ester, heteroaryl, heterocyclyl, lower alkyl, sulfone, sulfoxide, or hydroxyalkyl;

R5 independently represents hydrogen, carboxyl, amine, —NH—CO—NH—, —NH—CO—CH2—NH—,

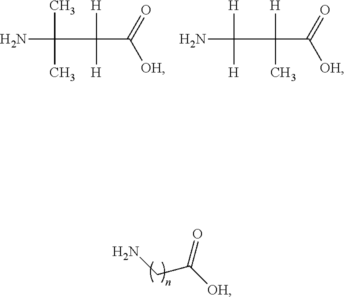

or an analog of any of the foregoing; where, n represents an integer from 0 to 11
R6 independently represents hydrogen, methyl, ethyl, butyl, amine, —NH—CO—, R—COO—R1, thiol, disulfide, or

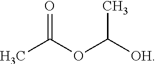

R7 independently represents 6,8-dithiooctanoic acid or its residue or its analog or its metabolite

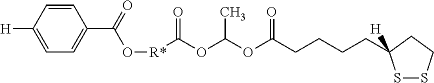

Wherein R* in some aspects represents

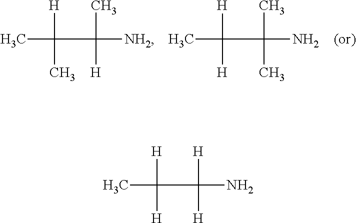

In certain embodiments, the present disclosure relates to the compounds and compounds of formula (I) or pharmaceutically acceptable salts thereof,

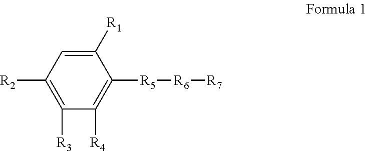

Wherein, R1, R2, R3 and R4 each independently represents hydrogen, methyl, amine, cyclohexyl methyl ether, butoxy, propoxy, halogen (Chlorine or Flourine), thiol, alkyl, alkyl thiol, acetyl thiol, disulfide, acyl, acylalkyl, alkenyl, alkylthioalkyl, alkynyl, alkoxyaryl, alkoxyalkyl, aryl, aralkyl, aryloxyalkyl, arylthioalkyl, cycloalkyl, ether, ester, heteroaryl, heterocyclyl, lower alkyl, sulfone, sulfoxide, or hydroxyalkyl;

R5 independently represents hydrogen, carboxyl, amine, —NH—CO—NH—, —NH—CO—CH2—NH—,

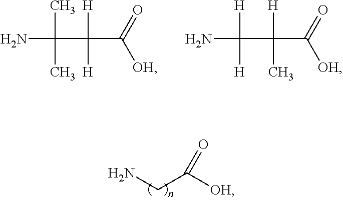

or an analog of any of the foregoing; where, n represents an integer from 0 to 11.
R6 independently represents hydrogen, amine, —NH—CO—, R—COO—R1, thiol, disulfide, or

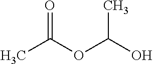

R7 independently represents 6,8-dithiooctanoic acid or its residue or its analog or its metabolite

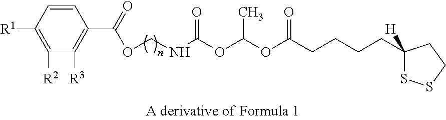

Wherein, in some aspects, R1, R2 and R3 is at least one of a hydrogen, methyl, ethyl, butyl, butoxy or propoxy; where, n represents an integer from 0 to 11.

In another embodiment, the final compound is

In one embodiment the pharmaceutically acceptable amount may be administered, but not limited to, as an injection. Other embodiments for administration may include peroral, topical, transmucosal, inhalation, targeted delivery and sustained release formulations.

Herein the application also provides a kit comprising any of the pharmaceutical compounds disclosed herein. The kit may comprise instructions for use in the treatment of pain or related complications.

The application also discloses a pharmaceutical compound comprising a pharmaceutically acceptable carrier and any of the compounds herein.

The compounds described herein have several uses. The present application provides, for example, methods of treating a patient suffering from pain manifested from chronic diseases or disorders, Hematological, Orthopedic, Cardiovascular, Renal, Skin, Neurological, Metastasis (cancer) or Ocular complications. The compounds may also be used in biochemical research, for example in studying and modulating neural voltage transmission and homeostasis and also neural channels.

### BRIEF DESCRIPTION OF DRAWINGS

FIG. 1 shows the method of synthesis of a final compound of formula 1.

FIG. 2 shows a 1H NMR analysis done for final compound

FIG. 2A shows an expanded version of a particular sector 1H NMR analysis of FIG. 2.

FIG. 3 shows 13C NMR test done for final compound.

FIG. 4 displays the drug application regiment for 21 days duration.

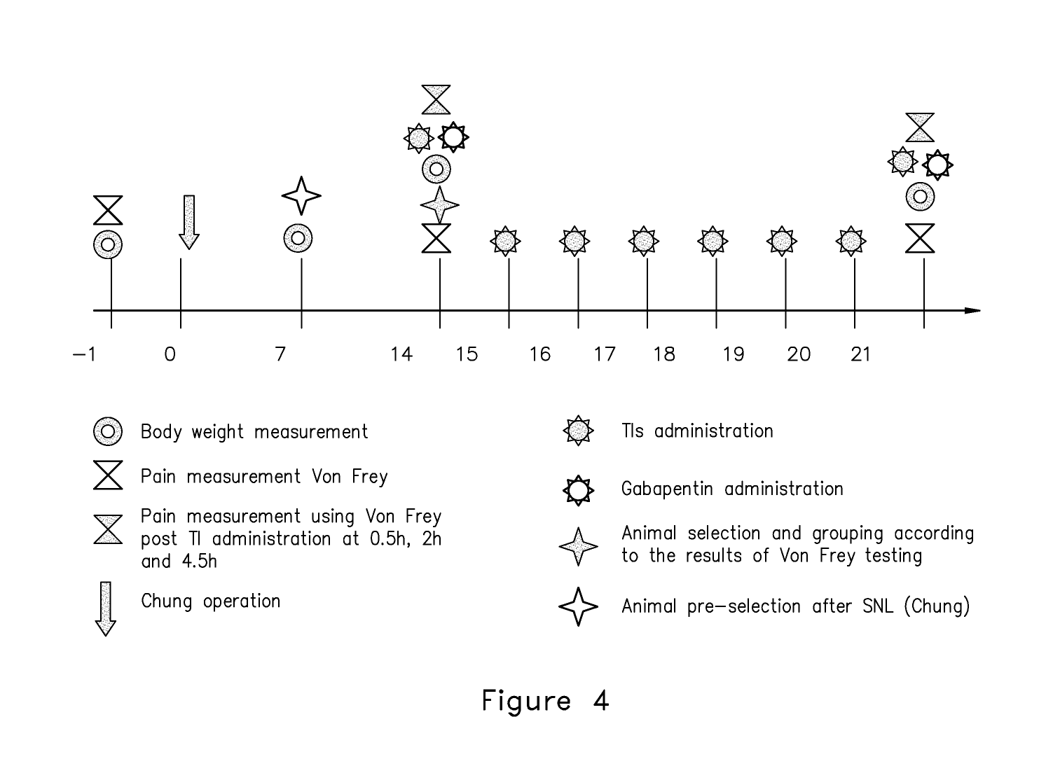

FIG. 5 displays comparative results of the compound, blank and the Gabapentin dose for pain induced rats.

### DETAILED DESCRIPTION

According to one embodiment, compound of formula 1 and its physiologically compatible acid-addition salts are used for the pharmaceutical preparations for the treatment and/or prophylaxis of pain, more specifically neuropathic pain.

### Definitions

As used herein, the following terms and phrases shall have the meanings set forth below. Unless defined otherwise, all technical and scientific terms used herein have the same meaning as commonly understood to one of ordinary skill in the art.

The term “alkyl” refers to the radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C1-C30 for straight chains, C3-C30 for branched chains), and more preferably 20 or fewer. Likewise, preferred cycloalkyls have from 3-10 carbon atoms in their ring structure, and more preferably have 5, 6 or 7 carbons in the ring structure.

The term “alkyl” as used herein refers to a saturated linear or branched-chain monovalent hydrocarbon radical of one to twelve carbon atoms. Examples of alkyl groups include, but are not limited to, methyl (Me, —CH3), ethyl (Et, —CH2CH3), 1-propyl (n-Pr, n-propyl, —CH2CH2CH3), 2-propyl (i-Pr, i-propyl, —CH(CH3)2), 1-butyl (n-Bu, n-butyl, —CH2CH2CH2CH3), 2-methyl-1-propyl (1-Bu, i-butyl, —CH2CH(CH3)2), 2-butyl (s-Bu, s-butyl, —CH(CH3)CH2CH3), 2-methyl-2-propyl (t-Bu, t-butyl, —C(CH3)3), 1-pentyl (n-pentyl, —CH2CH2CH2CH2CH3), 2-pentyl (—CH(CH3)CH2CH2CH3), 3-pentyl (—CH(CH2CH3)2), 2-methyl-2-butyl (—C(CH3)2CH2CH3), 3-methyl-2-butyl (—CH(CH3)CH(CH3)2), 3-methyl-1-butyl (—CH2CH2CH(CH3)2), 2-methyl-1-butyl (—CH2CH(CH3)CH2CH3), 1-hexyl (—CH2CH2CH2CH2CH2CH3), 2-hexyl (—CH(CH3)CH2CH2CH2 CH3), 3-hexyl (—CH(CH2CH3)(CH2CH2CH3)), 2-methyl-2-pentyl (—C(CH3)2CH2CH2CH3), 3-methyl-2-pentyl (—CH(CH3)CH(CH3)CH2CH3), 4-methyl-2-pentyl (—CH(CH3)CH2CH(CH3)2), 3-methyl-3-pentyl (—C(CH3)(CH2CH3)₂), 2-methyl-3-pentyl (—CH(CH2CH3)CH(CH3)2), 2,3-dimethyl-2-butyl (—C(CH3)2CH(CH3)2), 3,3-dimethyl-2-butyl (—CH(CH3)C(CH3)3, 1-heptyl, 1-octyl, and the like.

The term “alkenyl” refers to linear or branched-chain monovalent hydrocarbon radical of two to twelve carbon atoms with at least one site of unsaturation, i.e., a carbon-carbon, sp double bond, wherein the alkenyl radical includes radicals having “cis” and “trans” orientations, or alternatively, “E” and “Z” orientations. Examples include, but are not limited to, ethylenyl or vinyl (—CH═CH2), allyl (—CH2CH═CH2), and the like. The term “alkynyl” refers to a linear or branched monovalent hydrocarbon radical of two to twelve carbon atoms with at least one site of unsaturation, i.e., a carbon-carbon, sp triple bond. Examples include, but are not limited to, ethynyl (—C≡CH), propynyl (propargyl, —CH2C≡CH), and the like.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents, if not otherwise specified, can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF3, —CN and the like. Exemplary substituted alkyls are described below. Cycloalkyls can be further substituted with alkyls, alkenyls, alkoxys, alkylthios, aminoalkyls, carbonyl-substituted alkyls, —CF3, —CN, and the like.

The term “acyl” is art-recognized and refers to a group represented by the general formula hydrocarbylC(O)—, preferably alkylC(O)—.

“Aryl” means a monocyclic or polycyclic ring assembly wherein each ring is aromatic or when fused with one or more rings forms an aromatic ring assembly. If one or more ring atoms is not carbon (e.g., N, S), the aryl is a heteroaryl. Cx aryl and Cx-Y aryl are typically used where X and Y indicate the number of carbon atoms in the ring.

The term “acylamino” is art-recognized and refers to an amino group substituted with an acyl group and may be represented, for example, by the formula hydrocarbyl C(O)NH—.

The term “acylalkyl” is art-recognized and refers to an alkyl group substituted with an acyl group and may be represented, for example, by the formula hydrocarbyl C(O)alkyl.

The term “acyloxy” is art-recognized and refers to a group represented by the general formula hydrocarbylC(O)O—, preferably alkylC(O)O—.

The term “alkoxy” refers to an alkyl group, preferably a lower alkyl group, having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkenyl”, as used herein, refers to an aliphatic group containing at least one double bond and is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds.

Moreover, such substituents include all those contemplated for alkyl groups, as discussed below, except where stability is prohibitive. For example, substitution of alkenyl groups by one or more alkyl, carbocyclyl, aryl, heterocyclyl, or heteroaryl groups is contemplated.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “alkynyl”, as used herein, refers to an aliphatic group containing at least one triple bond and is intended to include both “unsubstituted alkynyls” and “substituted alkynyls”, the latter of which refers to alkynyl moieties having substituents replacing a hydrogen on one or more carbons of the alkynyl group. Such substituents may occur on one or more carbons that are included or not included in one or more triple bonds. Moreover, such substituents include all those contemplated for alkyl groups, as discussed above, except where stability is prohibitive. For example, substitution of alkynyl groups by one or more alkyl, carbocyclyl, aryl, heterocyclyl, or heteroaryl groups is contemplated.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The term “heteroalkyl”, as used herein, refers to a saturated or unsaturated chain of carbon atoms and at least one heteroatom, wherein no two heteroatoms are adjacent.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “heterocyclylalkyl”, as used herein, refers to an alkyl group substituted with a heterocycle group.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “ketone” is art-recognized and may be represented, for example, by the formula C(O)R9, wherein R9 represents a hydrocarbyl group.

The term “lower” when used in conjunction with a chemical moiety, such as, acyl, acyloxy, alkyl, alkenyl, alkynyl, or alkoxy is meant to include groups where there are ten or fewer non-hydrogen atoms in the substituent, preferably six or fewer. A “lower alkyl”, for example, refers to an alkyl group that contains ten or fewer carbon atoms, preferably six or fewer. Lower alkyls include methyl and ethyl. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The term “substituted” refers to moieties having substituents replacing hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this application, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

Unless specifically stated as “unsubstituted,” references to chemical moieties herein are understood to include substituted variants. For example, reference to an “aryl” group or moiety implicitly includes both substituted and unsubstituted variants.

“Substituted or unsubstituted” means that a given moiety may consist of only hydrogen substituents through available valencies (unsubstituted) or may further comprise one or more non-hydrogen substituents through available valencibs (substituted) that are not otherwise specified by the name of the given moiety. For example, isopropyl is an example of an ethylene moiety that is substituted by —CH3. In general, a non-hydrogen substituent may be any substituent that may be bound to an atom of the given moiety that is specified to be substituted. Examples of substituents include, but are not limited to, aldehyde, alicyclic, aliphatic, (Ci-io) alkyl, alkylene, alkylidene, amide, amino, aminoalkyl, aromatic, aryl, bicycloalkyl, bicycloaryl, carbamoyl, carbocyclyl, carboxyl, carbonyl group, cycloalkyl, cycloalkylene, ester, halo, heterobicycloalkyl, heterocycloalkylene, heteroaryl, heterobicycloaryl, heterocycloalkyl, oxo, hydroxy, iminoketone, ketone, nitro, oxaalkyl and oxoalkyl moieties, each of which may optionally also be substituted or unsubstituted. In one particular embodiment, examples of substituents include, but are not limited to, hydrogen, halo, nitro, cyano, thio, oxy, hydroxy, carbonyloxy, (Ci_io) alkoxy, (C4-12) aryloxy, hetero (Ci-io)aryloxy, carbonyl, oxycarbonyl, aminocarbonyl, amino, (Ci-10) alkylamino, sulfonamido, imino, sulfonyl, sulfinyl, (Ci-10) alkyl, halo (Ci-10) alkyl, hydroxy (Ci-10) alkyl, carbonyl (Ci-10) alkyl, thiocarbonyl (Ci_i0) alkyl, sulfonyl (Ci-10) alkyl, sulfinyl (Ci_io) alkyl, (Ci_i0) azaalkyl, imino (Ci-I0) alkyl, (C3-12) cycloalkyl (C1-5) alkyl, hetero (C3-12) cycloalkyl (Ci-I0) alkyl, aryl (Ci-I0) alkyl, hetero (Ci-10) aryl (C1-5) alkyl, (C9-12) bicycloaryl (Ci_s) alkyl, hetero (Ce-I2) bicycloaryl (Ci_{—}5) alkyl, (C3-12) cycloalkyl, hetero (C3-12) cycloalkyl, (C9-12) bicycloalkyl, hetero (C3-I2) bicycloalkyl, (C4-I2) aryl, hetero (Ci-10) aryl, (C9-12) bicycloaryl and hetero (C4-12) bicycloaryl. In addition, the substituent is itself optionally substituted by a further substituent. In one particular embodiment, examples of the further substituent include, but are not limited to, hydrogen, halo, nitro, cyano, thio, oxy, hydroxy, carbonyloxy, (Ci-10) alkoxy, (C4-I2) aryloxy, hetero (Ci-10) aryloxy, carbonyl, oxycarbonyl, aminocarbonyl, amino, (Ci-10) alkylamino, sulfonamido, imino, sulfonyl, sulfinyl, (Ci-10) alkyl, halo (Ci-10) alkyl, hydroxy (Ci-10) alkyl, carbonyl (Ci-10) alkyl, thiocarbonyl (Ci-10) alkyl, sulfonyl (Ci-10) alkyl, sulfinyl (Ci-10) alkyl, (Ci-10) azaalkyl, imino (Ci_io) alkyl, (C3-I2) cycloalkyl (Ci-5) alkyl, hetero (C3-12) cycloalkyl (Ci-10) alkyl, aryl (Ci_{—}10) alkyl, hetero (Ci-io) aryl (Ci_{—}5) alkyl, (C9-I2) bicycloaryl (C1-5) alkyl, hetero (C8-12) bicycloaryl (Ci_s) alkyl, (C3-12) cycloalkyl, hetero (C3_{—}12) cycloalkyl, (C9-12) bicycloalkyl, hetero (C3-12) bicycloalkyl, (C4-12) aryl, hetero (Ci-10) aryl, (C9-12) bicycloaryl and hetero (C4-12) bicycloaryl.

The compounds of the present disclosure can be present in the form of pharmaceutically acceptable salts. The compounds of the present disclosure can also be present in the form of pharmaceutically acceptable esters (i.e., the methyl and ethyl esters of the acids of formulal I to be used as prodrugs). The compounds of the present disclosure can also be solvated, i.e. hydrated. The solvation can be effected in the course of the manufacturing process or can take place i.e. as a consequence of hygroscopic properties of an initially anhydrous compound of formula 1 (hydration).

Compounds that have the same molecular formula but differ in the nature or sequence of bonding of their atoms or the arrangement of their atoms in space are termed “isomers.” Isomers that differ in the arrangement of their atoms in space are termed “stereoisomers.” Diastereomers are stereoisomers with opposite configuration at one or more chiral centers which are not enantiomers. Stereoisomers bearing one or more asymmetric centers that are non-superimposable mirror images of each other are termed “enantiomers.” When a compound has an asymmetric center, for example, if a carbon atom is bonded to four different groups, a pair of enantiomers is possible. An enantiomer can be characterized by the absolute configuration of its asymmetric center or centers and is described by the R- and S-sequencing rules of Cahn, Ingold and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “sulfate” is art-recognized and refers to the group OSO3H, or a pharmaceutically acceptable salt thereof.

As used herein, the term “pain” refers to an unpleasant sensory and emotional experience associated with actual or potential tissue damage caused by or resulting in stimulation of nociceptors in the peripheral nervous system, or by damage to or malfunction of the peripheral or central nervous systems and neural voltage channel transmission. Pain related diseases or disorders includes such as Cancer (chemotherapy and surgery related), Neurologic (bradykinesia, rigidity, tremor, ataxia, dyskinesia, dysarthria, seizures, neuropathic pain), Psychiatric (behavioral disturbances, cognitive impairment, psychosis), Ophthalmologic (dry eye, cataracts), Hematologic (haemolysis, coagulopathy), Renal (renal tubular defects, diminished glomerular filtration, nephrolithiasis), Cardiovascular (cardiomyopathy, arrhythmias, conduction disturbances, autonomic dysfunction), Musculoskeletal (osteomalacia, osteoporosis, degenerative joint diseases), Gastrointestinal (cholelithiasis, pancreatitis, bacterial peritonitis), Surgery or amputation related or any other medical condition, is well understood in the art, and includes administration of a compound which reduces the frequency of, or delays the onset of, symptoms of a medical condition in a subject relative to a subject which does not receive the compound.

The term “polymorph” as used herein is art-recognized and refers to one crystal structure of a given compound.

“Residue” is an art-recognized term that refers to a portion of a molecule. For instance, a residue of thioctic acid may be: dihydrolipoic acid, bisnorlipoic acid, tetranorlipoic acid, 6,8-bismethylmercapto-octanoic acid, 4,6-bismethylmercapto-hexanoic acid, 2,4-bismethylmeracapto-butanoic acid, 4,6-bismethylmercapto-hexanoic acid.

The phrases “parenteral administration” and “administered parenterally” as used herein refer to modes of administration other than enteral and topical administration, such as injections, and include without limitation intravenous, intramuscular, intrapleural, intravascular, intrapericardial, intraarterial, intrathecal, intracapsular, intraorbital, intracardiac, intradennal, intraperitoneal, transtracheal, subcutaneous, subcuticular, intra-articular, subcapsular, subarachnoid, intraspinal and intrasternal injection and infusion.

A “patient,” “subject,” or “host” to be treated by the subject method may mean either a human or non-human animal, such as primates, mammals, and vertebrates.

The phrase “pharmaceutically acceptable” is art-recognized. In certain embodiments, the term includes compositions, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” is art-recognized, and includes, for example, pharmaceutically acceptable materials, compositions or vehicles, such as a liquid or solid filler, diluent, solvent or encapsulating material involved in carrying or transporting any subject composition, from one organ, or portion of the body, to another organ, or portion of the body. Each carrier must be “acceptable” in the sense of being compatible with the other ingredients of a subject compound and not injurious to the patient. In certain embodiments, a pharmaceutically acceptable carrier is non-pyrogenic. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) cocoa butter and suppository waxes; (9) oils, such as peanut oil, cottonseed oil, sunflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “polymorph” as used herein is art-recognized and refers to one crystal structure of a given compound.

The term “prodrug” is intended to encompass compounds that, under physiological conditions, are converted into the therapeutically active agents of the present disclosure. A common method for making a prodrug is to include selected moieties that are hydrolyzed under physiological conditions to reveal the desired molecule. In other embodiments, the prodrug is converted by an enzymatic activity of the host animal.

The term “prophylactic or therapeutic” treatment is art-recognized and includes administration to the host of one or more of the subject compounds. If it is administered prior to clinical manifestation of the unwanted condition (e.g., disease or other unwanted state of the host animal) then the treatment is prophylactic, i.e., it protects the host against developing the unwanted condition, whereas if it is administered after manifestation of the unwanted condition, the treatment is therapeutic, (i.e., it is intended to diminish, ameliorate, or stabilize the existing unwanted condition or side effects thereof).

The term “treating” is art-recognized and includes preventing a disease, disorder or condition from occurring in an animal which may be predisposed to the disease, disorder and/or condition but has not yet been diagnosed as having it; inhibiting the disease, disorder or condition, e.g., impeding its progress; and relieving the disease, disorder, or condition, e.g., causing regression of the disease, disorder and/or condition. Treating the disease or condition includes ameliorating at least one symptom of the particular disease or condition, even if the underlying pathophysiology is not affected, such as treating the pain of a subject by administration of an analgesic agent even though such agent does not treat the cause of the pain. The term “treating”, “treat” or “treatment” as used herein includes curative, preventative (e.g., prophylactic), adjunct and palliative treatment.

The phrase “therapeutically effective amount” is an art-recognized term. In certain embodiments, the term refers to an amount of a salt or compound disclosed herein that produces some desired effect at a reasonable benefit/risk ratio applicable to any medical treatment. In certain embodiments, the term refers to that amount necessary or sufficient to eliminate or reduce medical symptoms for a period of time. The effective amount may vary depending on such factors as the disease or condition being treated, the particular targeted constructs being administered, the size of the subject, or the severity of the disease or condition. One of ordinary skill in the art may empirically determine the effective amount of a particular compound without necessitating undue experimentation.

In certain embodiments, the pharmaceutical compositions described herein are formulated in a manner such that said compounds will be delivered to a patient in a therapeutically effective amount, as part of a prophylactic or therapeutic treatment. The desired amount of the compound to be administered to a patient will depend on absorption, inactivation, and excretion rates of the drug as well as the delivery rate of the salts and compounds from the subject compounds. It is to be noted that dosage values may also vary with the severity of the condition to be alleviated. It is to be further understood that for any particular subject, specific dosage regimens should be adjusted over time according to the individual need and the professional judgment of the person administering or supervising the administration of the compositions.

Additionally, the optimal concentration and/or quantities or amounts of any particular salt or compound may be adjusted to accommodate variations in the treatment parameters. Such treatment parameters include the clinical use to which the preparation is put, e.g., the site treated, the type of patient, e.g., human or non-human, adult or child, and the nature of the disease or condition.

The term “solvate” as used herein, refers to a compound formed by solvation (e.g., a compound formed by the combination of solvent molecules with molecules or ions of the solute).

When used with respect to a pharmaceutical composition or other material, the term “sustained release” is art-recognized. For example, a subject compound which releases a substance over time may exhibit sustained release characteristics, in contrast to a bolus type administration in which the entire amount of the substance is made biologically available at one time. For example, in particular embodiments, upon contact with body fluids including blood, spinal fluid, mucus secretions, lymph or the like, one or more of the pharmaceutically acceptable excipient may undergo gradual or delayed degradation (e.g., through hydrolysis) with concomitant release of any material incorporated therein, e.g., an therapeutic and/or biologically active salt and/or compound, for a sustained or extended period (as compared to the release from a bolus). This release may result in prolonged delivery of therapeutically effective amounts of any of the therapeutic agents disclosed herein.

The phrases “systemic administration,” “administered systemically,” “peripheral administration” and “administered peripherally” are art-recognized, and include the administration of a subject compound, therapeutic or other material at a site remote from the disease being treated. Administration of an agent directly into, onto, or in the vicinity of pain sensation of the disease being treated, even if the agent is subsequently distributed systemically, may be termed “local” or “topical” or “regional” administration, other than directly into the central nervous system, e.g., by subcutaneous administration, such that it enters the patient's system and, thus, is subject to metabolism and other like processes.

The present disclosure also contemplates prodrugs of the compounds disclosed herein, as well as pharmaceutically acceptable salts of said prodrugs.

Generally, in carrying out the methods detailed in this application, an effective dosage for the compounds of Formulas 1 is in the range of about 0.3 mg/kg/day to about 60 mg/kg/day in single or divided doses, for instance 1 mg/kg/day to about 50 mg/kg/day in single or divided doses. The compounds of Formulas 1 may be administered at a dose of, for example, less than 2 mg/kg/day, 5 mg/kg/day, 10 mg/kg/day, 20 mg/kg/day, 30 mg/kg/day, or 40 mg/kg/day. Compounds of Formula 1 may also be administered to a human patient at a dose of, for example, between 50 mg and 1000 mg, between 100 mg and 800 mg, or less than 1000, 900, 800, 700, 600, 500, 400, 300, 200, or 100 mg per day. In certain embodiments, the compounds herein are administered at an amount that is less than 95%, 90%, 80%, 70%, 60%, 50%, 40%, 30%, 20%, or 10% of the compound of formula 1 required for the same therapeutic benefit.

In some cases, it may be desirable to administer in the form of a kit, it may comprise a container for containing the separate compounds such as a divided bottle or a divided foil packet. Typically the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

Compound of formula lis disclosed as follows: In one embodiment, a compound of formula 1 is disclosed.

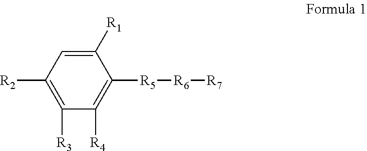

Wherein, R1, R2, R3 and R4 each independently may represent hydrogen, methyl, amine, cyclohexyl methyl ether; butoxy, propoxy, halogen (Chlorine or Flourine), thiol, alkyl, alkyl thiol, acetyl thiol, disulfide, acyl, acylalkyl, alkenyl, alkylthioalkyl, alkynyl, alkoxyaryl, alkoxyalkyl, aryl, aralkyl, aryloxyalkyl, arylthioalkyl, cycloalkyl, ether, ester, heteroaryl, heterocyclyl, lower alkyl, sulfone, sulfoxide, or hydroxyalkyl;
R5 independently represents hydrogen, carboxyl, amine, —NH—CO—NH—, —NH—CO—CH2—NH—,

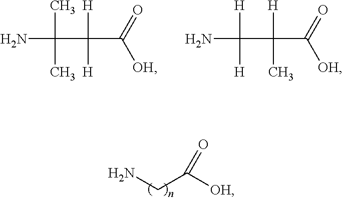

or an analog of any of the foregoing; where, n represents an integer from 0 to 11.
R6 independently represents hydrogen, amine, —NH—CO—, R—COO—R1, thiol, disulfide, or

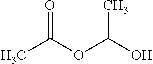

R7 independently represents at least one of a 6,8-dithiooctanoic acid,

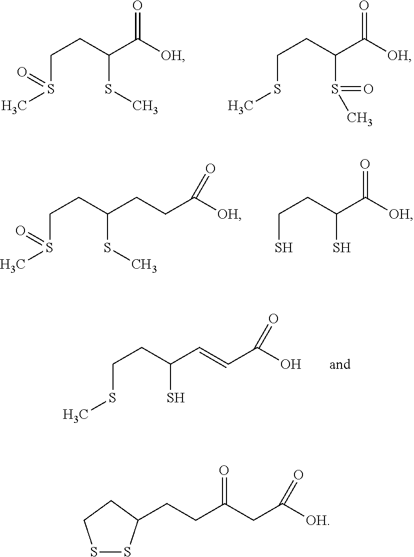

In one embodiment, R1, R2, R3 or R4 may represent a hydrogen, methyl, amine, butoxy, propoxy, chlorine or

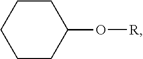

R5 represents —NH—CO—NH—, —NH—CO—CH2—NH— or —COO—(CH2)—NH—, R6 represents

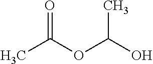

and R7 represents R-isomer of residue or analog or derivative or metabolite of 6,8-dithiooctanoic acid.

In certain embodiments, the compounds and compounds of formula (I) or pharmaceutically acceptable salts thereof,

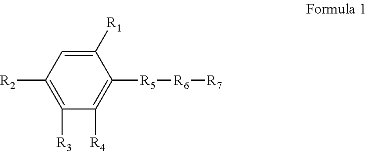

Wherein, R1, R2, R3 and R4 each independently represents hydrogen, methyl, amine, cyclohexyl methyl ether, butoxy, propoxy, halogen (Chlorine or Flourine), thiol, alkyl, alkyl thiol, acetyl thiol, disulfide, acyl, acylalkyl, alkenyl, alkylthioalkyl, alkynyl, alkoxyaryl, alkoxyalkyl, aryl, aralkyl, aryloxyalkyl, arylthioalkyl, cycloalkyl, ether, ester, heteroaryl, heterocyclyl, lower alkyl, sulfone, sulfoxide, or hydroxyalkyl;

R5 independently represents hydrogen, carboxyl, amine, —NH—CO—NH—, —NH—CO—CH2—NH—,

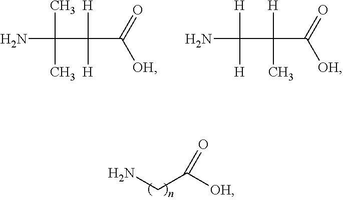

or an analog of any of the foregoing; where, n represents an integer from 0 to 11
R6 independently represents hydrogen, methyl, ethyl, butyl, amine, —NH—CO—, R—COO—R1, thiol, disulfide, or

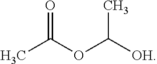

R7 independently represents 6,8-dithiooctanoic acid or its residue or its analog or its metabolite

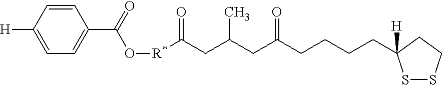

Wherein R* in some aspects represents

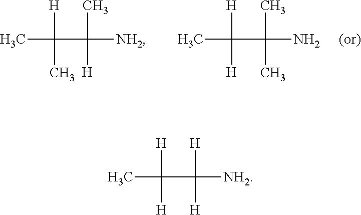

In certain embodiments, the present disclosure relates to the compounds and compounds of formula (1) or pharmaceutically acceptable salts thereof,

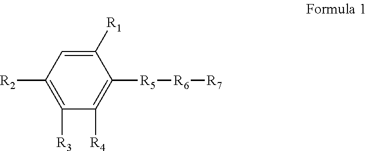

Wherein, R1, R2, R3 and R4 each independently represents hydrogen, methyl, amine, cyclohexyl methyl ether, butoxy, propoxy, halogen (Chlorine or Flourine), thiol, alkyl, alkyl thiol, acetyl thiol, disulfide, acyl, acylalkyl, alkenyl, alkylthioalkyl, alkynyl, alkoxyaryl, alkoxyalkyl, aryl, aralkyl, aryloxyalkyl, arylthioalkyl, cycloalkyl, ether, ester, heteroaryl, heterocyclyl, lower alkyl, sulfone, sulfoxide, or hydroxyalkyl;

R5 independently represents hydrogen, carboxyl, amine, —NH—CO—NH—, —NH—CO—CH2—NH—,

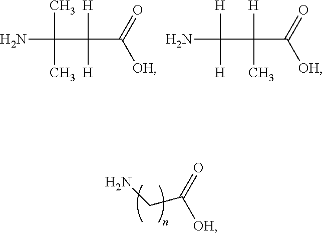

or an analog of any of the foregoing; where, n represents an integer from 0 to 11.
R6 independently represents hydrogen, amine, —NH—CO—, R—COO—R1, thiol, disulfide, or

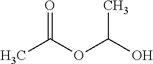

R7 independently represents 6,8-dithiooctanoic acid or its residue or its analog or its metabolite

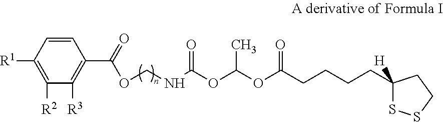

Wherein, in some aspects, R1, R2 and R3 is at least one of a hydrogen, methyl, ethyl, butyl, butoxy or propoxy; where, n represents an integer from 0 to 11.

### Method of Synthesis of the Compound

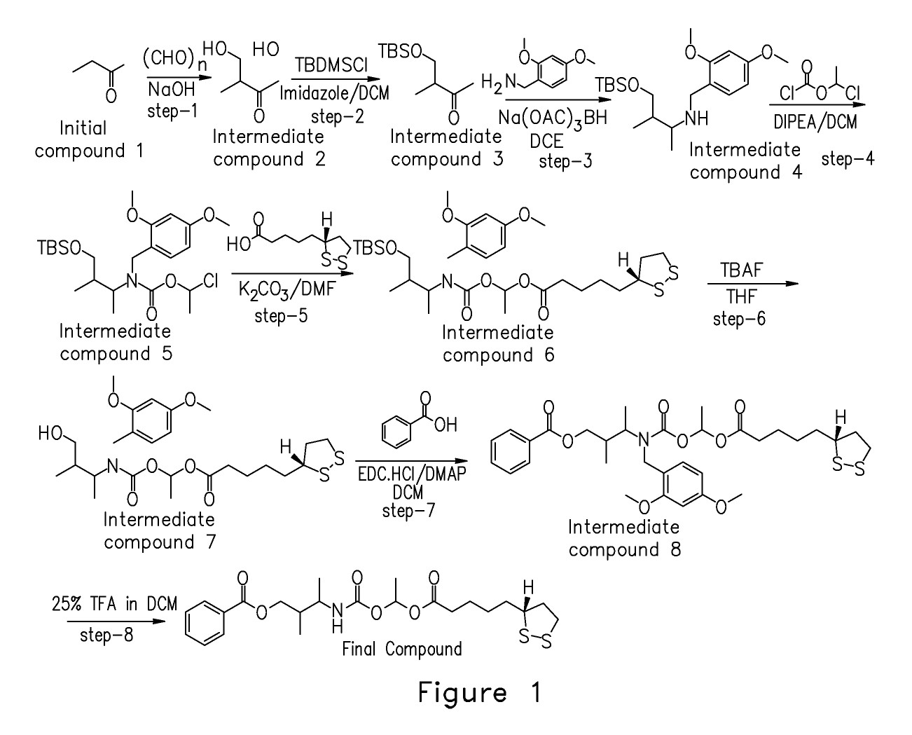

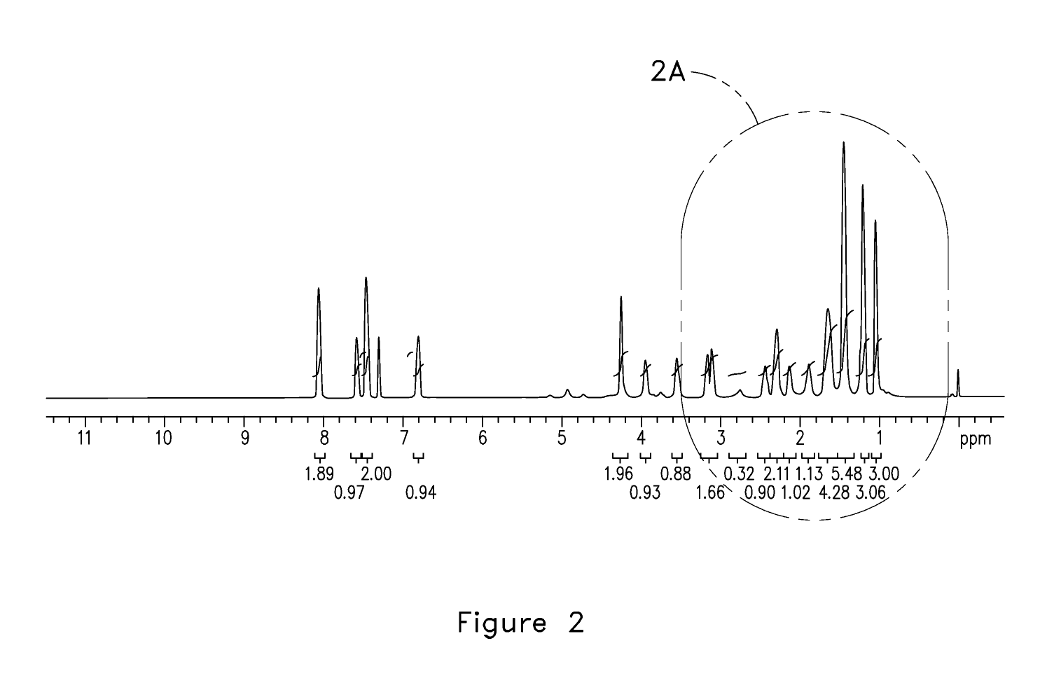

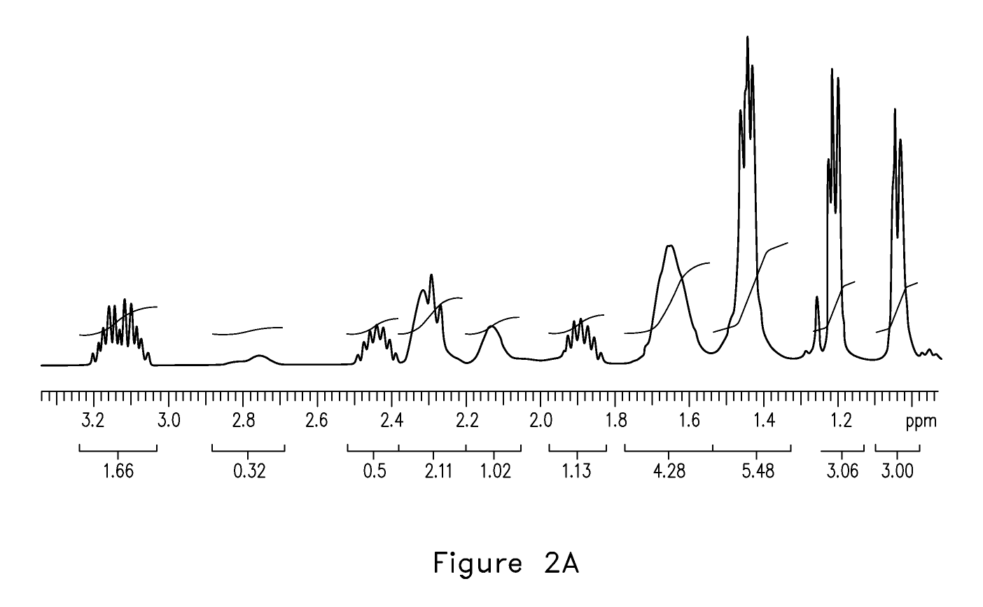

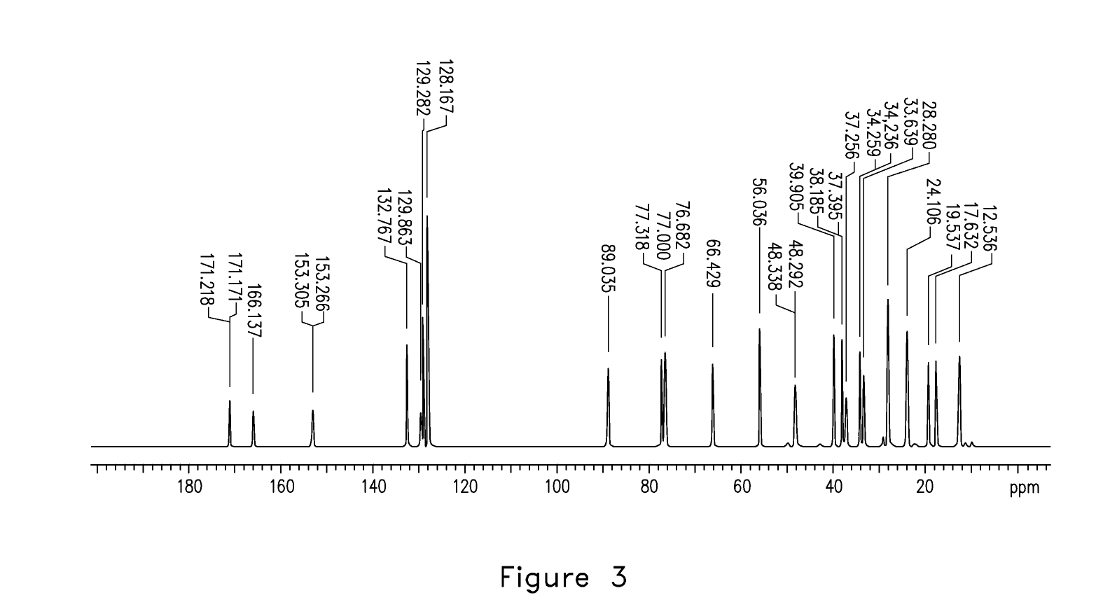

FIG. 1 discloses the following steps in a diagramatic format showing the steps, intermediate compounds and reactants. FIGS. 2 and 3 show a analysis of compounds using standard analytical methods.

### STEP-1: Synthesis of 4-hydroxy-3-methyl-2-butanone

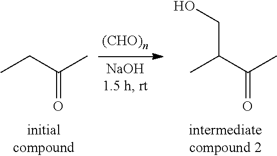

Ethyl methyl ketone (450 ml; reactant as well as solvent) and paraformaldehyde (30.0 g) at 40° C. were mixed and were continuously stirred. NaOH solution (1.0 M aq) was slowly added drop by drop until the reaction mixture turned blue. After 40 min, the two layers were observed to be visibly separated indicating all the solid paraformaldehyde was dissolved. The reaction mixture further neutralized by drop-wise addition of glacial acetic acid and then extracted with chloroform. The organic layer was separated, washed with brine, dried over anhydrous Na₂SO₄ and evaporated under reduced pressure. The intermediate compound 2 (shown as 2) was purified by high vacuum distillation under reduced pressure to yield 26.0 g of ketone (intermediate compound 2) as yellow oil. The yellow oil had a boiling point (bp) of 90-110° C. at 12 mm Hg. (Yield: 25.7%, calculated based on paraformaldehyde)

TABLE 1
¹H NMR (CDCl₃, 400 MHz)

| | splitting pattern | | |
|---|---|---|---|
| δ | & J value | Protons | Group |
| 3.64-3.75 | m | 2H | CH₂OH |
| 2.70-2.79 | m | 1H | CH |
| 2.19 | s | 3H | CH₃ |
| 1.15-2.10 | d, J = 4.8 Hz | 3H | CH₃CH |

### STEP-2: Synthesis of Intermediate Compound 3

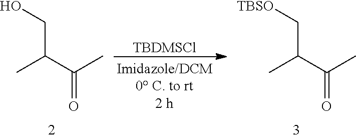

To a solution of 4-hydroxy-3-methyl-2-butanone (1.0 eq) in DCM (100 ml; LR grade), imidazole (2.5 eq) was added, followed by tert-Butyldimethylsilyl chloride (1.5 eq) in DCM (20 ml; LR grade) at 0° C. The reaction mixture was allowed to stir for 2 h at room temperature. On completion of the reaction (monitored by TLC), the reaction mixture was diluted with DCM (100 ml), washed with water (2×100 ml) followed by brine solution (100 ml), dried over anhydrous Na₂SO₄ and evaporated under reduced pressure. The crude residue intermediate compound 3 was purified by column chromatography over 100-200 mesh silica gel by using 5% ethyl acetate-pet ether as eluent to yield 8.4 g (79%) of intermediate compound 3 (shown as 3) a pale yellow as oil.

TABLE 2
¹H NMR (CDCl₃, 400 MHz)

| | splitting pattern | | |
|---|---|---|---|
| δ | & J value | Protons | Group |
| 3.61-3.75 | m | 2H | CH₂OTBS |
| 2.69-2.75 | m | 1H | CH |
| 2.18 | s | 3H | CH₃ |
| 1.01-1.03 | d, J = 0.8 Hz | 3H | CH₃CH |
| 0.85 | s | 9H | tBuSi |
| 0.01-0.02 | s | 6H | MeSiMe |

### STEP-3: Synthesis of Intermediate Compound 4

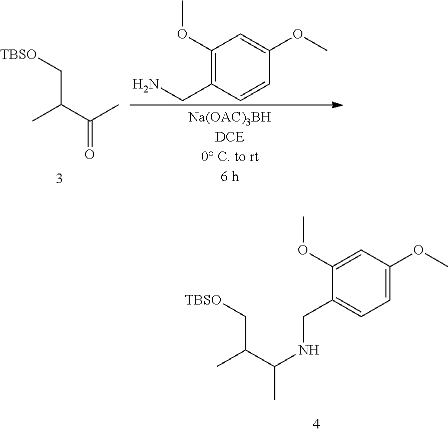

Intermediate compound 3 (1.0 eq) and 2,4-dimethoxy benzyl amine (1.0 eq) were mixed in 1,2 Dichloroethane (100 ml; LR grade) and stirred for 2 h at room temperature, then sodium triacetoxy borohydride (1.2 eq) was added followed by acetic acid (1.5 eq) at 0° C. and the reaction mixture was allowed to stir for 4-6 h at rt. On completion of the reaction (monitored by TLC), the reaction mixture was diluted with DCM (100 ml), washed with water (2×100 ml) followed by brine solution (100 ml), dried over anhydrous Na₂SO₄ and evaporated under reduced pressure to get crude product as viscous oil which was purified by column chromatography over neutral alumina by using 30% ethyl acetate-pet ether as eluent to yield 5.0 g (60%) of intermediate compound 4 (shown as 4) as a pale yellow liquid.

TABLE 3
¹H NMR (CDCl₃, 400 MHz)

| | Splitting pattern | | |
|---|---|---|---|
| δ | & J value | Protons | Group |
| 7.12-7.15 | d, J = 1.2 Hz | 1H | ArH |
| 6.40-6.43 | m | 2H | ArH |
| 3.77-3.80 | s | 6H | 2 x OMe |
| 3.62-3.74 | m | 4H | CH₂OTBS, CH₂Ar |
| 3.48-3.17 | m | 1H | CH₃CHN |
| 2.70-2.74 | m | 1H | CH₃CH |
| 1.04 | d, J = 1.2 Hz | 3H | CH₃ |
| 0.89-0.84 | m | 12H | tBuSi, CH₃ |
| 0.02 | s | 6H | MeSiMe |

### STEP-4: Synthesis of Intermediate Compound 5 and 6

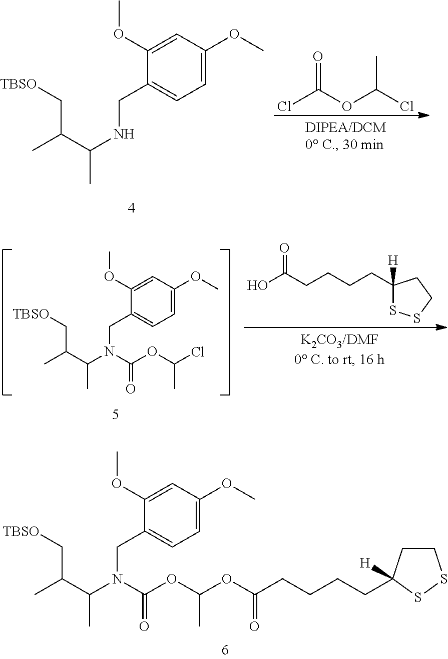

To a solution of intermediate compound 4 (1.0 eq) in dry DCM (100 ml), N,N-diisopropylethylamine (1.5 eq) was added, followed by drop-wise addition of 1-chloroethylchloroformate (1.2 eq) at 0° C. and the reaction mixture was allowed to stir for 30 min at 0° C. On completion of the reaction (monitored by TLC), the reaction mixture containing intermediate compound 5 (shown as 5) was directly used for the next step, without any further purification.

To the above reaction mixture lipoic acid (1.2 eq) and anhydrous K₂CO₃ (3.0 eq) in dry dimethylformamide (DMF) (50 ml) at 0° C. was slowly added. The reaction mixture was allowed to stir for 16 h at room temperature (rt). Reaction was monitored by TLC. On completion of the reaction, reaction mixture was poured into 100 ml of distilled water and extracted with EtOAc (2×100 ml). The combined organic layers were washed with brine solution (100 ml), dried over anhydrous Na₂SO₄ and evaporated under reduced pressure. The resultant crude was purified by column chromatography over 100-200 mesh silica gel by using 10% ethyl acetate-pet ether as eluent to yield 3.0 g (35%) of intermediate compound 6 as a yellow liquid.

TABLE 4
¹H NMR (CDCl₃, 400 MHz)

| | Splitting pattern | | |
|---|---|---|---|
| δ | & J value | Protons | Group |
| 7.18-7.07 | m | 1H | OCHO |
| 6.85-6.90 | m | 1H | ArH |
| 6.44-6.42 | m | 2H | ArH |
| 4.32-4.38 | m | 2H | NCH₂Ar |
| 3.78, 3.79 | s | 6H | 2 x OMe |
| 3.52-3.74 | m | 3H | CH₂OTB, CHS |
| 3.09-3.18 | m | 2H | CH₂S |
| 2.24-2.56 | m | 4H | CH₃CHN, CH, CH₂CO |
| 1.99-1.88 | m | 2H | CH₂ |
| 1.42-1.78 | m | 9H | 3x CH2, CH3 |
| 1.02-1.20 | m | 6H | 2XCH₃ |
| 0.89 | m | 9H | tBuSi, |
| 0.02 | s | 6H | MeSiMe |

### STEP-5: Synthesis of Intermediate Compound 7

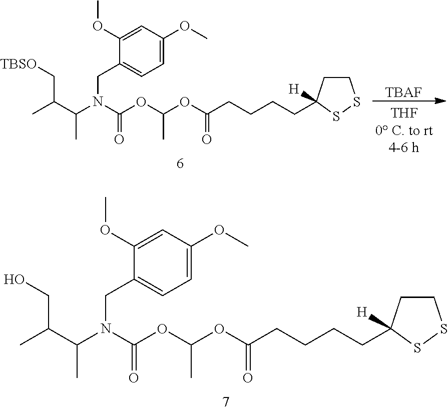

To a solution of intermediate compound 6 (1.0 eq) in dry TETRAHYDROFURAN (THF) (20 ml), tetrabutylammonium fluoride (1M solution in THF) (3.0 eq) was added drop-wise at 0° C. and the reaction mixture was allowed to stir for 4-6 h at rt. Reaction was monitored by TLC. On completion of the reaction, the reaction mixture was poured into water (50 ml) and extracted with EtOAc (2×50 ml), the combined organic layers were washed with brine solution (100 ml), dried over anhydrous Na₂SO₄ and evaporated under reduced pressure. The crude was purified by column chromatography over 100-200 mesh silica gel by using 25% ethyl acetate-pet ether as eluent to yield 0.18 g (23%) of intermediate compound 7 as yellow oil.

TABLE 5
¹H NMR (CDCl₃, 400 MHz), Mass (m/z): m/z = 530.67 [M + H]⁺

| | Splitting pattern | | |
|---|---|---|---|
| δ | & J value | Protons | Group |
| 6.78-7.01 | m | 2H | OCHO, ArH |
| 6.42-6.44 | m | 2H | ArH |
| 4.10-4.40 | m | 3H | NCH₂Ar, NCHCH3 |
| 3.80-3.79 | ss | 6H | 2 x OMe |
| 3.40-3.55 | m | 3H | CH₂OH, CHS |
| 3.04-3.19 | m | 2H | CH₂S |
| 2.21-2.45 | m | 3H | CH₃CH, CH₂CO |
| 1.85-1.95 | m | 2H | CH₂ |
| 1.20-1.64 | m | 9H | 3x CH2, CH3 |
| 1.18 | d, J = 8.0 Hz | 3H | CH₃ |
| 0.87 | d, J = 8.0 Hz | 3H | CH₃ |

### STEP-6: Synthesis of Intermediate Compound 8

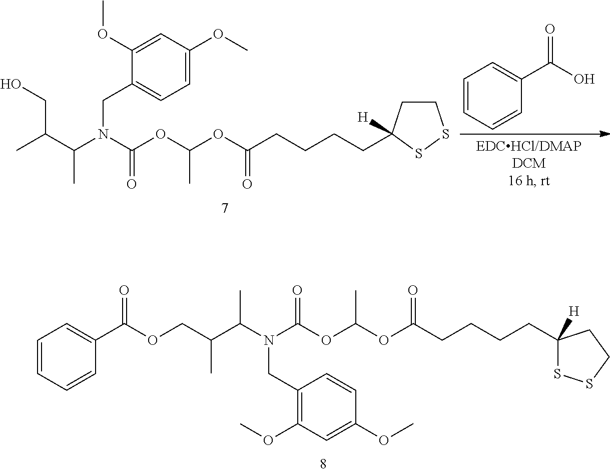

Intermediate compound 7 (1.0 eq) and benzoic acid (1.0 eq) was mixed and was kept in a stirred state. DCM (500 ml; LR grade), EDC.HCl (1.2 eq) and DMAP (1.2 eq) were added at room temperature to the above reaction mixture and the solution was stirred for another 16 h at room temperature. The progress and completion of the reaction was monitored by TLC. On completion of the reaction, the reaction mixture was diluted with DCM (200 ml), washed with water (2×300 ml) followed by brine solution (300 ml) and dried over anhydrous Na₂SO₄ and evaporated under reduced pressure. The resultant crude was purified by column chromatography over 100-200 mesh silica gel by using 15% ethyl acetate-pet ether as eluent to yield 10.0 g (68.58%) of intermediate compound 8 as a pale yellow gum.

TABLE 6
¹H NMR (CDCl₃, 400 MHz)

| | Splitting pattern | | |
|---|---|---|---|
| δ | & J value | Protons | Group |
| 8.02 | d = 8.0 Hz | 2H | PhH |
| 7.52-7.57 | m | 1H | PhH |
| 7.42-7.46 | m | 2H | PhH |
| 7.07.7.11 | m | 1H | OCHO |
| 6.86-6.93 | m | 1H | ArH |
| 6.44-6.42 | m | 2H | ArH |
| 4.17-4.42 | m | 4H | CH₂O, NCH₂Ar |
| 3.95 | m | 1H | CHN |
| 3.80-3.79 | ss | 6H | 2 x OMe |
| 3.52-3.58 | m | 1H | CHS |
| 3.07-3.21 | m | 2H | CH₂S |
| 2.25-2.44 | m | 4H | CH₂, CH CH₂CO |
| 1.86-1.91 | m | 1H | CH |
| 1.42-1.66 | m | 9H | 3x CH₂, CH₃ |
| 1.14-1.19 | m | 3H | CH₃ |
| 0.96-1.02 | m | 3H | CH₃ |

### STEP-7: Synthesis of Final Compound

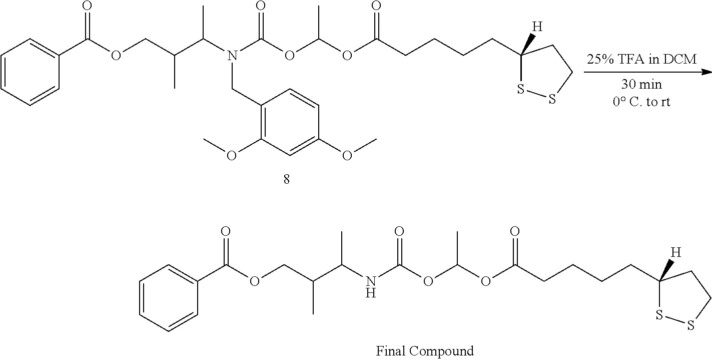

25% TFA in DCM (150 ml) was added to compound 8 (1.0 eq) at 0° C. and the reaction mixture was allowed to stir for 30 min at the same temperature. Reaction was monitored by TLC. On completion of the reaction, the saturated Na₂CO₃ solution (100 ml) was added to make it basic and extracted with DCM (200 ml). It was further dried over anhydrous Na₂SO₄ and evaporated under reduced pressure. The crude was purified by column chromatography over neutral alumina (Merck) by using 30% ethyl acetate-pet ether as eluent to yield 4.7 g (51.6%) of final compound as a pale yellow gummy solid.

TABLE 7
¹H NMR (CDCl₃ 400 MHz)

| | splitting pattern | | |
|---|---|---|---|
| δ | & J value | Protons | Group |
| 8.02 | d = 7.2 Hz | 2H | PhH |
| 7.55-7.57 | m | 1H | PhH |
| 7.43-7.47 | m | 2H | PhH |
| 6.80 | m | 1H | OCHO |
| 4.78 | m | 1H | NH |
| 4.25 | d = 6.0 Hz | 2H | CH₂OBz |
| 3.93 | m | 1H | CHN |
| 3.53 | m | 1H | CHS |
| 3.08-3.14 | m | 2H | CH₂S |
| 2.42 | m | 1H | CH |
| 2.22-2.38 | m | 2H | CH₂CO |
| 2.15 | m | 1H | CH₂ |
| 1.88 | m | 1H | CH₂ |
| 1.60-1.68 | m | 4H | 2xCH₂ |
| 1.40-1.54 | m | 5H | CH₂, CH₃ |
| 1.2 | d = 3.2 Hz | 3H | CH₃ |
| 0.96-1.02 | d = 3.2 Hz | 3H | CH₃ |

TABLE 8
¹³C NMR (CDCl₃, 400 MHz) - Final compound

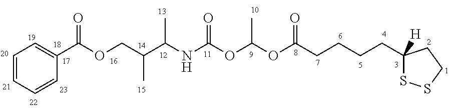

| δ | Carbon position | Group |
|---|---|---|
| 171.21 | C8 | CO |
| 166.13 | C17 | PhCO |
| 153.30 | C11 | NCO |
| 132.76 | C21 | PhCH |
| 129.86 | C18 | PhC |
| 129.28 | C19, C23 | PhCH |
| 128.16 | C20, C22 | PhCH |
| 89.03 | C9 | OCHO |
| 66.42 | C16 | CH₂OBz |
| 56.03 | C12 | CHN |
| 48.33 | C3 | CHS |
| 39.90 | C2 | CH2 |
| 38.10 | C14 | CH |
| 37.39 | C1 | CH₂S |
| 34.25 | C7 | CH₂CO |
| 34.23 | C4 | CH₂ |
| 28.28 | C5 | CH₂ |
| 24.10 | C6 | CH₂ |
| 19.53 | C13 | CH₃ |
| 17.63 | C10 | CH₃ |
| 12.53 | C15 | CH₃ |

### Method of Treatment, Testing and Results Using the Final Compound

Experimental animals were male SD rats with a starting weight of 230-250 grams. Total number of animals were n=90 for surgery and n=60 after selection. The animals were caged in groups of 3 in a temperature and humidity controlled area. They were maintained on a 12 hr light/dark cycle and had ad libitum access to food and water.

Neuropathic pain inducement was done by following principles of Chung induced model. The SD rats were anesthetized using ketamine/xylazine sodium. The rats were shaved and placed in prone position for surgery. The L5-L6 spinal nerves were surgically litigated. The rats were returned to their cages for recuperation and recovery under comfortable warm conditions using heat lamps.

After seven days of surgery a pre selection was performed. Animals that indicate signs of post operative pain were to be selected to proceed. Pain is detected by observing when one or more of the criteria are met as follows. Licking of the operated paw, accompanied by gentle biting or pulling on the nails with the mouth; placing the leg in the air; bearing weight on the side contra-lateral to the nerve injury; deformities of the hind paw and abnormal posture and walking; weakness of the left hind paw. The animals that exhibited these pain occurring symptoms were chosen for further steps. However, the animal must be able to move its leg to ensure that the L₄ spinal nerve is intact. If the animal cannot move its leg, it was excluded from the study.

Second level of selection of the rats was done on day 14 after the surgery. Von Frey test was performed on the preselected rats after day 7 on day 14. Using Von Frey methodology, animals with a pain threshold of ≦26 g for the operated leg will be included in the study. After this selection step, the animals were randomly placed into their experimental groups.

Blank, positive control and test compound: Blank was just the medium used for dissolving other compounds. The positive control was Gabapentin and the test compound was the final compound of formula 1 discussed in the instant disclosure. Three types of experimental groups were formed. Three different types of chemicals were used to determine the efficacy of the chemicals as well comparison of the instant disclosed compound with a positive control was performed. The final compound of the instant application was administered at 100 mg/kg and 150 mg/kg body weight as two different groups. The Gabapentin was administered at 150 mg/kg body weight.

TABLE 9
Test Groups and dose regiment:

| Group | Group | | | Dose | Volume | | |
|---|---|---|---|---|---|---|---|
| No. | Size | Test Item | Route | (mg/kg) | (ml/kg) | Dosing Regime | Testing Regime |
| 1 | N = 10 | Blank | IP | 0 mg/kg | 5 ml/kg | Once daily starting | Von Frey testing at 0.5 |
| | | | | | | on study day 14 | hours, at 2 hours and at |
| | | | | | | through study day | 4.5 hours after dosing on |
| | | | | | | 21 | study days 14 and 21 |
| 2 | N = 10 | KRB-2/Pre | IP | 150 mg/kg | 5 ml/kg | Once daily starting | Von Frey testing at 0.5 |
| | | | | | | on study day 14 | hours, at 2 hours and at |
| | | | | | | through study day | 4.5 hours after dosing on |
| | | | | | | 21 | study days 14 and 21 |
| 3 | N = 10 | Gabapentin | IP | 150 mg/kg | 5 ml/kg | On study days 14 | Von Frey testing at 0.5 |
| | | (Positive | | | | and 21 | hours, at 2 hours and at |
| | | Control) | | | | 2 hours prior to | 4.5 hours after dosing on |
| | | | | | | Von Frey testing | study days 14 and 21 |
| 4 | N = 10 | KRB-2/Pre | IP | 100 mg/kg | 5 ml/kg | Once daily starting | Von Frey testing at 0.5 |
| | | | | | | on study day 14 | hours, at 2 hours and at |
| | | | | | | through study day | 4.5 hours after dosing on |
| | | | | | | 21 | study days 14 and 21 |

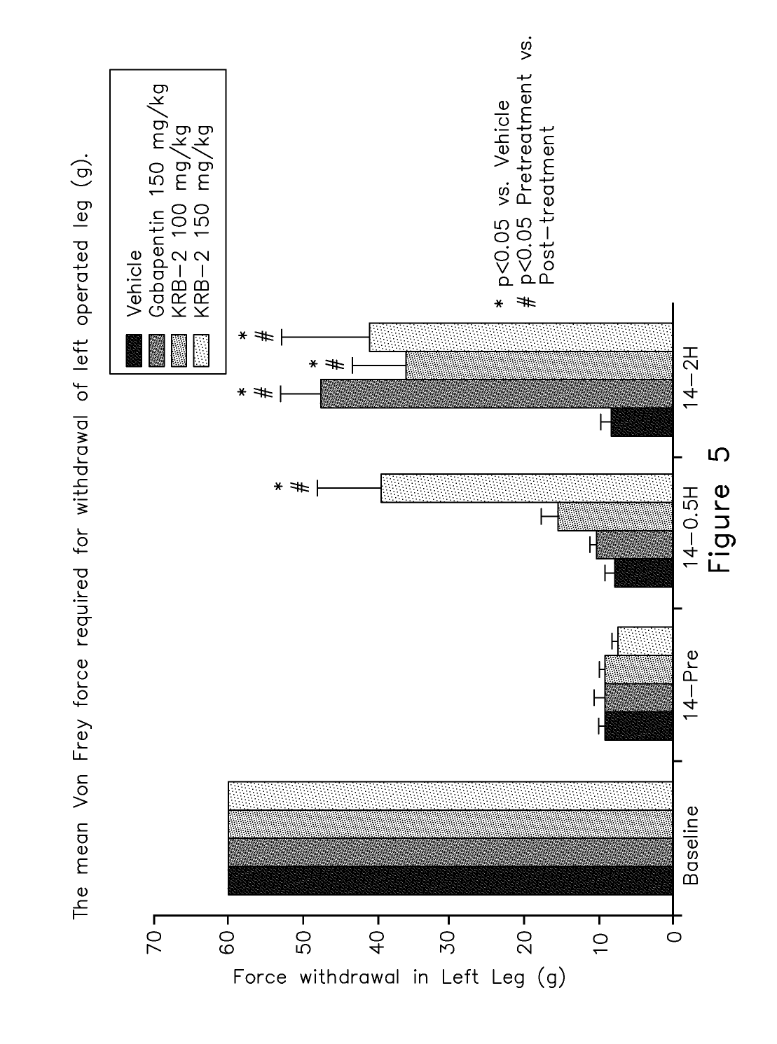

As shown in FIG. 5, on day 14 blank compound and final compound will be administered once daily starting on study day 14 through study day 21. Gabapentin, the positive control, will be administered 2 hours prior to pain testing on study days 14 and 21. The Von Frey test will be performed prior to final compound administration (pre-final compound injection) and after final compound administration at 0.5 hours, 2 hours and 4.5 hours post-final compound injection on study days 14 and 21. Additionally, if the Von Frey response for the operated leg of one of the six treatment groups is significant at 4.5 hours, all the groups will be tested again at 6.5 hours. In all instances, unless decided otherwise in the course of the study, all dosing solutions are applied as once a day intra peritoneal (IP) administration on each of the repeated dosing sessions. After the termination of the study the animals were euthanized.

A one-way ANOVA following by a Tukey post-test was performed to determine significance of treatment effects compared to the blank. A p value <0.05 is considered to represent a significant difference.

As shown in FIG. 5, the instant final compound of formula 1 at a dose of 150 mg/kg was effective in treating the spinal nerve ligation model for neuropathic pain in rats as reflected in the parameters of mechanical allodynia at 2 hours post-treatment on study days 14 and 21. The activity of the instant final compound of formula 1 at a dose of 150 mg/kg was similar to the activity of Gabapentin, the positive control in this study.

The present disclosure provides among other things compound, method to synthesize the compound for formula 1 and treating pain in mammals using the compound of formula 1. While specific embodiments of the subject disclosure have been discussed, the above specification is illustrative and not restrictive. Many variations of the compounds, compounds and methods herein will become apparent to those skilled in the art upon review of this specification.

### INDUSTRIAL APPLICABILITY

There are multiple applications for compound of formula 1, compound of formula 1 with pharmaceutically acceptable additives to treat mammals suffering from pain, more specifically neuropathic pain in general. These compounds may be used in the treatment of diseases related to pain and its related complications.

What is claimed is:

1. A compound, comprising:
a compound of formula 1: 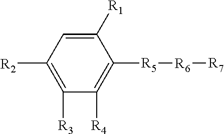
wherein R¹, R², R³ and R⁴ are at least one of hydrogen, methyl, amine, cyclohexyl methyl ether, butoxy, propoxy, halogen (chlorine, flourine), thiol, alkyl, alkyl thiol, acetyl thiol, disulfide, acyl, acylalkyl, alkenyl, alkylthioalkyl, alkynyl, alkoxyaryl, alkoxyalkyl, aryl, aralkyl, aryloxyalkyl, arylthioalkyl, cycloalkyl, ether, ester, heteroaryl, heterocyclyl, lower alkyl, sulfone, sulfoxide and hydroxyalkyl;
R⁵ represents at least one of a hydrogen, carboxyl, amine, —NH—CO—NH—, —NH—CO—CH2—NH—, 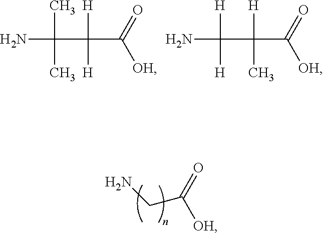
and an analog of any one of the foregoing; wherein n represents an integer from 0 to 11;
R⁶ represents at least one of a hydrogen, amine, —NH—CO—, R—COO—R¹, thiol, disulfide and 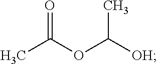
and
R⁷ represents at least one of a 6,8-dithiooctanoic acid,

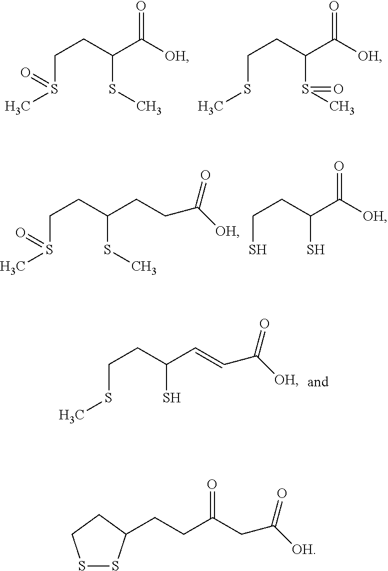

2. The compound of claim 1, wherein R¹, R², R³ and R⁴ represents at least one of a hydrogen, methyl, amine, butoxy, propoxy, chlorine and 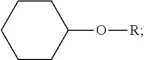
R⁵ represents at least one of —NH—CO—NH—, —NH—CO—CH2—NH— and —COO—(CH2)—NH—;
R⁶ represents 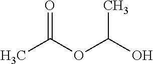
and R⁷ represents at least one of a R-isomer of residue, analog, derivative and a metabolite of 6,8-dithiooctanoic acid.

3. The compound of claim 2, further comprising;
a final compound with a formula of:

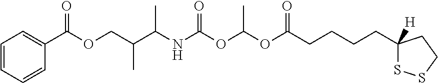

4. The compound of claim 3, further comprising of a pharmaceutical composition comprising at least one of hydrate, a solvate, a mesylate and a hydrochloride salt.

5. A method, comprising;
synthesizing a compound of formula 1, further comprising:
stirring a reaction mixture of ethyl methyl ketone and paraformaldehyde at 40° C.;
slowly adding NaOH until the reaction mixture turns blue;
neutralizing the reaction mixture by adding glacial acetic acid;
separating and washing an organic layer with brine;
drying the organic layer over anhydrous Na₂SO₄,
evaporating the organic layer under reduced pressure to form a dried product; and
purifying the dried product by high vacuum distillation under reduced pressure to yield an intermediate compound 2.

6. The method of claim 5, further comprising:
adding 4-hydroxy-3-methyl-2-butanone dissolved in DCM, imidazole followed by tert-Butyldimethylsilyl chloride in DCM at 0° C. to the intermediate compound 2;
stirring a reaction mixture for 2 hours at room temperature;
monitoring the completion of the reaction by using thin layer chromatography;
diluting the reaction mixture with DCM;
washing the reaction mixture with water and then by brine solution;
drying the reaction mixture over anhydrous Na₂SO₄;
evaporating the resultant reaction mixture under reduced pressure to form a crude residue;
purifying the crude residue by column chromatography over 100-200 mesh silica gel by using 5% ethyl acetate-pet ether as eluent to yield an intermediate compound 3 as pale yellow as oil.

7. The method of claim 6, further comprising;
mixing the intermediate compound 3, and 2,4-dimethoxy benzyl amine in 1,2 Dichloroethane and creating a reaction mixture;
stirring the reaction mixture for 2 hours at room temperature;
adding then sodium triacetoxy borohydride to the reaction mixture followed by acetic acid at 0° C.;
stirring the reaction mixture for 4 to 6 hours at room temperature;
diluting the reaction mixture with DCM;
washing the reaction mixture with water followed by brine solution;
drying the reaction mixture over anhydrous Na₂SO₄ and evaporating under reduced pressure to get a crude product as viscous oil; and
purifying the crude product by column chromatography over neutral alumina by using 30% ethyl acetate-pet ether as eluent to yield an intermediate compound 4 as a pale yellow liquid.

8. The method of claim 7, further comprising;
adding N,N-diisopropylethylamine to a solution of the intermediate compound 4 in dry DCM forming a reaction mixture;
adding drop-wise 1-chloroethylchloroformate at 0° C. to the reaction mixture;
stirring the reaction mixture for 30 min at 0° C.;
monitoring the completion of the reaction by TLC; and
using the reaction mixture containing an intermediate compound 5 for the next step.

9. The method of treatment of claim 8, further comprising:
adding to the above reaction mixture containing an intermediate compound 5 lipoic acid and anhydrous in dry DMF at 0° C.;
stirring the reaction mixture for 16 h at room temperature;
mixing 100 ml of distilled water to the reaction mixture and extracting with EtOAc to create a combined organic layer;
washing the combined organic layer with brine solution;
drying the combined organic layer over anhydrous Na₂SO₄ and evaporating under reduced pressure to form a crude; and
purifying the crude by column chromatography over 100-200 mesh silica gel by using 10% ethyl acetate-pet ether as eluent to yield an intermediate compound 6 as a yellow liquid.

10. The method of claim 9, further comprising:
adding tetrahydrofuran (THF) and tetrabutylammonium fluoride drop-wise to a solution of intermediate compound 6 at 0° C. to create a reaction mixture;
stirring the reaction mixture for 4-6 h at room temperature;
pouring the reaction mixture into water and extracting with ethyl acetate(EtAOC) to form a combined organic layer;
washing the combined organic layer with brine solution;
drying the combined organic layer over anhydrous Na₂SO₄ and evaporating under reduced pressure to form a crude; and
purifying the crude by column chromatography over 100-200 mesh silica gel by using 25% ethyl acetate-pet ether as eluent to yield an intermediate compound 7 as yellow oil.

11. The method of claim 10, further comprising:
mixing and stirring the intermediate compound 7 and benzoic acid to form a reaction mixture;
adding dichloromethane(DCM), 1-[3-(Dimethylamino)propyl]-3-ethylcarbodiimide hydrochloride (EDC.HCl) and 4-dimethylaminopyridine (DMAP) at room temperature to the reaction mixture;
stirring the reaction mixture for another 16 h at room temperature;
monitoring the progress of the reaction by using TLC;
diluting the reaction mixture with DCM;
washing the reaction mixture with water followed by brine solution;
drying the reaction mixture over anhydrous Na₂SO₄ and evaporating under reduced pressure to form a crude; and
purifying the crude by column chromatography over 100-200 mesh silica gel by using 15% ethyl acetate-pet ether as eluent to yield an intermediate compound 8 as a pale yellow gum.

12. The method of claim 11, further comprising:
adding 25% TFA in DCM the intermediate compound 8 at 0° C. to form a reaction mixture;
stirring the reaction mixture for 30 min at the same temperature;
adding the saturated Na₂CO₃ solution to make the reaction mixture basic and extracting with DCM;
drying the reaction mixture over anhydrous Na₂SO₄ and evaporating under reduced pressure; and
purifying the reaction mixture by column chromatography over neutral alumina by using 30% ethyl acetate-pet ether as eluent to yield a final compound as a pale yellow gummy solid.

13. A method of treating, comprising;
administering the compound of formula 1, comprising: 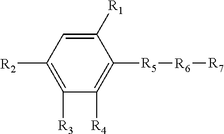
wherein, R1, R2, R3 and R4 each independently may represent hydrogen, methyl, amine, cyclohexyl methyl ether, butoxy, propoxy, halogen (Chlorine or Flourine), thiol, alkyl, alkyl thiol, acetyl thiol, disulfide, acyl, acylalkyl, alkenyl, alkylthioalkyl, alkynyl, alkoxyaryl, alkoxyalkyl, aryl, aralkyl, aryloxyalkyl, arylthioalkyl, cycloalkyl, ether, ester, heteroaryl, heterocyclyl, lower alkyl, sulfone, sulfoxide, or hydroxyalkyl;
R5 independently represents hydrogen, carboxyl, amine, —NH—CO—NH—, —NH—CO—CH2—NH—, 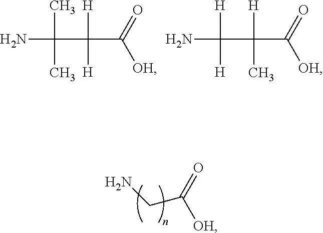
or an analog of any of the foregoing; where, n represents an integer from 0 to 11.
R6 independently represents hydrogen, amine, —NH—CO—, R—COO—R1, thiol, disulfide, or 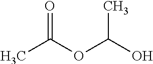
R7 independently represents at least one of a 6,8-dithiooctanoic acid,

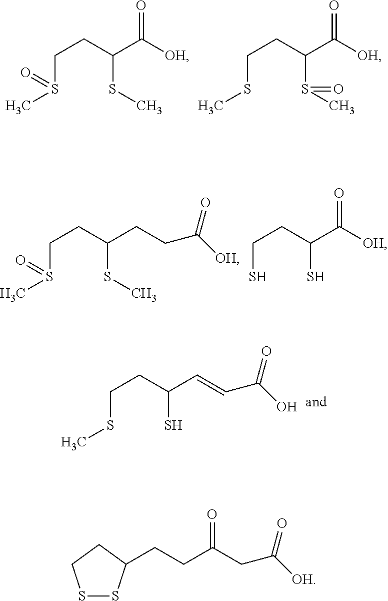

14. The method of treatment of claim 13; wherein the final compound is

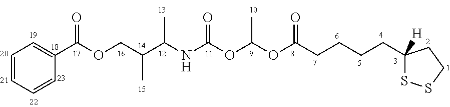

15. The method of treatment of claim 13, further comprising:
injecting the mammal with a dosage of 1-200 mg/kg body weight.

16. The method of treatment of claim 13, wherein administering is one of non-invasive peroral, topical, enteral, transmucosal, targeted delivery, sustained release delivery, delayed release, pulsed release and parenteral methods.

17. The method of treatment of claim 13, further comprising:
administering the compound of formula 1 in a day at least one of a single time and multiple times.

18. The method of treatment of claim 16, wherein administering is intra peritoneal injection.
